(19) United States
(12) Patent Application Publication
(10) Pub. No.: US 2026/0245740 A1
(43) Pub. Date: Aug. 20, 2026

(54) AUGMENTED-INTELLIGENCE BASED PERSONALIZED VIRTUAL PATIENT SIMULATOR

(71) Applicants: The University of Chicago, Chicago, IL (US); Mayo Foundation for Medical Education and Research, Jacksonville, FL (US); University College London, London (GB); National Laboratory for Scientific Computing, Petrópolis (BR)

(72) Inventors: Jonathan S. Grinstein, Chicago, IL (US); Pablo J. Blanco, Rio de Janeiro (BR); Ryo Torii, London (GB); Rohan Goswami, Jacksonville, FL (US)

(21) Appl. No.: 19/471,234

(22) PCT Filed: Mar. 29, 2024
(86) PCT No.: PCT/US2024/022340

§ 371 Date: Sep. 30, 2025

Related U.S. Application Data
(60) Provisional application No. 63/456,270, filed on Mar. 31, 2023.

(51) Int. Cl. G16H 50/50; G06N 20/20; G16H 10/60; G16H 50/70

(52) U.S. Cl. CPC G16H 50/50; G06N 20/20; G16H 10/60; G16H 50/70

(57) ABSTRACT

A system, comprising at least one cardiac sensor adapted to measure a hemodynamic profile of a patient; and a computing node operatively coupled to the cardiac sensor and configured to perform the steps of reading a hemodynamic profile of a patient; based on the hemodynamic profile, tuning a plurality of parameters of a cardiovascular model to create a digital twin of the patient; augmenting the hemodynamic profile of the patient with at least one parameter generated from the digital twin; providing the augmented hemodynamic profile to a pretrained machine learning model and receiving therefrom a patient profile; and outputting the patient profile for clinical decision support.

### RELATED APPLICATION

This application claims the benefit of U.S. Provisional Application No. 63/456270, filed on Mar. 31, 2023. The entire teachings of the above application(s) are incorporated herein by reference.

### BACKGROUND OF THE INVENTION

An estimated 6.7 million Americans have heart failure, of which approximately 180,000 have advanced heart failure. For patients living with advanced heart failure, heart replacement therapy in the form of a left ventricular assist device (LVAD) or cardiac transplantation are the gold standard. Unfortunately, the vast majority of medically eligible patients with advanced heart failure never receive these life extending therapies, given that only ~7,000 patients in the United States undergo heart replacement therapy each year. While some of this divide is related to limitations in the availability of donor hearts, the vast majority of patients who are medically suitable for heart replacement therapy are either never referred to tertiary care centers with expertise in LVAD or transplant or are referred too late, at which point they may no longer be acceptable candidates. The window in which patients remain candidates for advanced heart failure care is small, early referral and optimization of patients is paramount to improving outcomes and optimizing survival.

All patients with advanced heart failure have unique trajectories with variable rates of disease progression, medication tolerance, and survival. Often the warning signs of accelerated disease can be subtle and easily missed in the early stages of the disease process. As heart failure worsens, the clinical picture often accelerates, and patients progress into deeper stages of cardiogenic shock. This has life-ending potential if not appropriately identified and stabilized with early institution of intravenous medications and mechanical circulatory support (MCS) devices. For patients in cardiogenic shock, every hour delay in stabilization with an appropriate MCS device increases mortality by 10%.

Diagnostic testing can aid in risk stratification and identify patients with heart failure who have progression of their disease necessitating advanced therapies (e.g. iv medication, transplant, or LVAD). In addition to the clinical course and laboratory data, cardiopulmonary exercise testing (CPET) and an invasive hemodynamic assessment during a right heart catheterization (RHC) are often used to help stratify at risk patients with heart failure. While these tools do provide additive prognostic value, the threshold values which are often used to denote increased risk are often ill-defined (i.e. cardiac index<1.8 L/min/m2 vs 2.0 L/min/m2 vs. 2.2 L/min/m2; pulmonary capillary wedge pressure>15 mmHg vs 18 mmHg; Peak VO2<14 mL/kg/min vs 12 mL/kg/min). For certain variables, discrete thresholds may exist, however, these thresholds are defined at the population level and are statistical in nature, thus often have limited prognostic potential for the individual patient in question.

### SUMMARY OF THE INVENTION

In an example embodiment, the present invention is a method, comprising: reading a hemodynamic profile of a patient; based on the hemodynamic profile, tuning a plurality of parameters of a cardiovascular model to create a digital twin of the patient; augmenting the hemodynamic profile of the patient with at least one parameter generated from the digital twin; providing the augmented hemodynamic profile to a pretrained machine learning model and receiving therefrom a patient profile; and outputting the patient profile for clinical decision support.

In another example embodiment, the present invention is a system, comprising: at least one cardiac sensor adapted to measure a hemodynamic profile of a patient; and a computing node operatively coupled to the cardiac sensor and configured to perform a method according to any one of the example embodiments of the present invention.

In yet another example embodiment, the present invention is a computer program product for clinical decision support, the computer program product comprising a computer readable storage medium having program instructions embodied therewith, the program instructions executable by a processor to cause the processor to perform a method according to any of the example embodiments of the present invention.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1A and FIG. 1B schematically depict an embodiment of the VPA platform described herein.

FIG. 2 is a schematic diagram of a closed-loop compartmental representation of the cardiovascular parameters (CVS).

FIG. 3 is a schematic representation of a full hemodynamic profile generated by the closed-loop model shown in FIG. 2 for different LVAD working conditions. Energetic profile processed from the model output is characterized through PV-loops, and blood volume distribution.

FIG. 4 is a schematic diagram of a parameter identification procedure according to an example embodiment that finds the optimal model parameters that make the model to predict a HP that matches the patient HP.

FIG. 5 is a schematic representation of training and validation of an ML model, featuring input variables (patient hemodynamic profile) and endpoint patient outcome.

FIG. 6 is a schematic representation of integration of VPS and ML4HF technologies. Patient HP is augmented using the CVS model to find variables that characterize the hidden energetic state of the patient. The pair HP/EP is fed to the ML model to predict patient outcome (PO).

FIG. 7 is a schematic representation of a SoC architecture of an exemplary embodiment of the present invention.

FIG. 8 is a schematic diagram of a computer node that can be employed by exemplary embodiments of the present invention.

### DETAILED DESCRIPTION OF THE INVENTION

A description of example embodiments of the invention follows.

The pressure volume (P-V) loop is a diagnostic tool that allows for a nuanced assessment of the individual patients' energetic and physiologic profile. This data helps define when a patient transitions from a favorable coupled energetic state (i.e. stable state) to a less favorable uncoupled state (i.e. unstable state) when the ability of the heart to eject blood and/or the ability of the systemic circulation to accept blood becomes impaired. An uncoupled ventricular-arterial unit has unfavorable myocardial energetics due to a drop in mechanical efficiency. This decline in energetics represents a clear transition point of a patient with worsening advanced heart failure. While the P-V loop remains the gold standard to define the energetic state of the heart, practical considerations including the cost of catheters and equipment, technical nature of data acquisition and lack of catheter stability (or operator experience) limits its use almost exclusively for research purposes.

Described herein is a Virtual Patient Simulator (VPS), a novel diagnostic tool that incorporates both in vivo clinical data as well as in silico modeling of a patients' physiologic and energetic profile to help triage and manage patients with advanced heart failure and cardiogenic shock. Using patient-specific data from the hemodynamic assessment, the modeling first starts by reverse engineering the patient-specific P-V loop. Next, a machine learning model for Heart Failure patients (ML4HF) is employed which receives two inputs: 1) parameters derived from the compartmental model; and 2) parameters directly measured from the patient. As an optional additional level of patient-specific data on cardiac energetics, blood from patients can also be sampled for future metabolomic analysis, and the metabolomic profile can be integrated into the model. Unlike other machine learning algorithms which only use traditional variables including demographic data, laboratory data and standard hemodynamic variables, the integration of the VPS with the ML4HF (VPS-ML4HF) adds an additional layer of predictive accuracy to understand the timeline of an individual likelihood for acute heart failure events, potential for myocardial recovery and risk of disease progression despite application of standardized medical therapy. Accordingly, in various embodiments, cardiac energetics and PV loop-specific coupling ratios are combined with clinical variables directly measured and abstracted from the patient and related medical testing.

The VPS-ML4HF platform can be used to dichotomize patients with advanced heart failure by identifying key inflection points during progression of, and recovery from, their diseased state.

The heart failure spectrum encompasses functional and clinical stages. The model disclosed herein utilizes the clinical and hemodynamic data to combine both accepted systems, the ACC stage of heart failure as well as the SCAI stage of progressive shock (“A→E”). As patients decline and are likely to have worsening prognostic findings based on clinical testing, they move from Stage A to Stage E. Often, however, patients are too sick when in Stage E to be considered for advanced heart failure options such as LVAD or heart transplantation.

Utilization of VPS-ML4HF as a platform streamlines the early detection, intervention, and optimization of patients both in the outpatient and inpatient settings.

For ambulatory patients who are progressing to Stage D (end-stage), advanced heart failure, the VPS-ML4HF platform can define appropriate time for: medication titration, device-based intervention, and referral to tertiary centers. This strategy can guide the anticipated trajectory and urgency of the evaluation (i.e. inpatient vs outpatient evaluation) in addition to projecting a patient specific profile that outlines acuity of illness, aggressiveness of needed therapy, and likely outcome (transplant, LVAD, recovery, or death)

For patients admitted to the hospital with cardiogenic shock (SCAI C->E), the VPS-ML4HF platform can guide appropriate stabilization strategies, outline recovery potential, and integrate with multi-modality testing available to those that are critically ill (i.e. helping to determine which support device or IV medications to use, as well as suggesting escalation and de-escalation strategies).

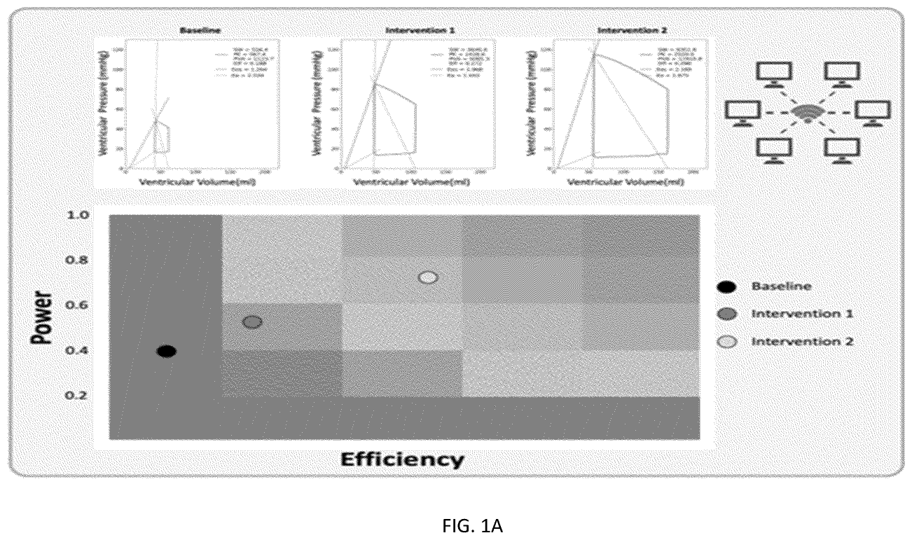

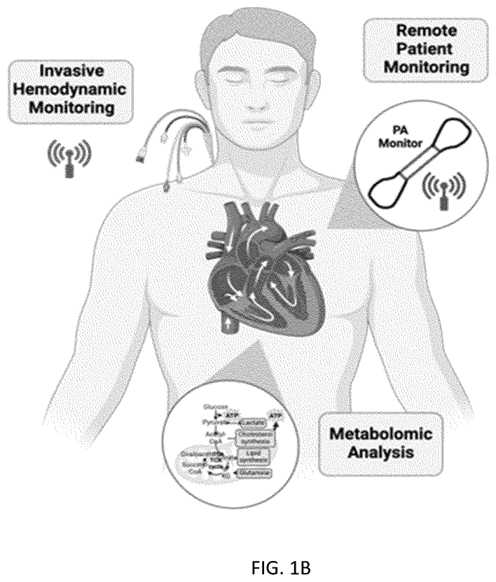

Selection of an intervention for a given patient is schematically depicted in FIG. 1A and FIG. 1B. FIG. 1A schematically represents how one of two possible interventions can be chosen based on the representation of a patient's ventricular pressure as a function of ventricular volume, translated into a representation of the patient on a power/efficiency plane. The data for the determination of the intervention is gathered, for example, by a computer node network and is based on various patient's parameters measured directly as shown in FIG. 1B.

The VPS platform relies on a closed-loop compartmental representation of the cardiovascular system relies upon a linear model of the connected intra and extra cardiac components of the cardiopulmonary circulatory loop to describe the hemodynamic states in each system. These compartments stand for the systemic and pulmonary arterial districts and venous pools, the four cardiac chambers and valves. In addition, mechanical support devices such as durable or temporary LVADs can be incorporated as additional (active) elements (compartments) in the model. In addition to the example provided herein, another suitable model is provided in Blood flow modeling under LVAD physiology. From global circulation to local hemodynamics, https://doi.org/10.1101/2022.01.28.478161, which is hereby incorporated by reference. Another suitable model is provided in A Closed-Loop Modeling Framework for Cardiac-to-Coronary Coupling, https://www.ncbi.nlm.nih.gov/pmc/articles/PMC8919076/, which is hereby incorporated by reference. More generally, suitable models: 1) model entire circulation (i.e., closed-loop) such that they capture interaction between different parts of the circulation (e.g., coupling between right ventricle and pulmonary vascular tree); and 2) have a representation of different local regions (i.e., compartmental) that can reflect characteristics of the local part via model parameters of that particular region. These two factors together facilitate calibration of the model to each individual patient so to construct the VPS.

Within each compartment, physical laws are satisfied, namely mass and momentum conservation laws. These laws are written in terms of ordinary differential equations, which characterize the state of the compartment as a function of time. In each compartment, blood pressure (P), blood flow rate (Q), and blood volume (V) represent the main state variables.

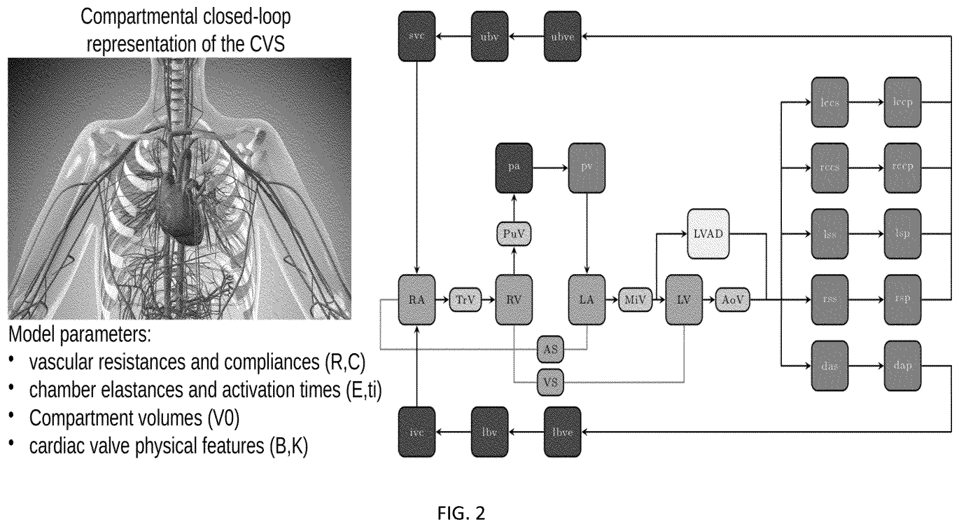

FIG. 2 depicts a schematic diagram of a closed-loop representation where the arterial system is compartmentalized into different peripheral districts, featured as red boxes, and venous pools are split into upper and lower body segments consisting of small and large venous districts, displayed in blue boxes. Cardiac chambers (orange boxes) are coupled through cardiac valves (light blue boxes). Atrial and ventricular coupling between right and left heart sides is represented through lime boxes. As per the abbreviations, in this illustrative example we have: RA, LA, RV, LV: right/left atria, right/left ventricles, AS, VS: atrial septum, ventricular septum; TrV, PuV, MiV, AoV: tricuspid, pulmonary, mitral, aortic valves; pa, pv: pulmonary arteries/veins; LVAD: left ventricular assist device; lccs, recs, lccp, rccp: left/right common carotid systemic and left/right common carotid peripheral; Iss, rss, lsp, rsp: left/right subclavian systemic and left/right subclavian peripheral; das, dap: descending aorta systemic, descending aorta peripheral; ubve, lbve, ubv, lbv, svc, ivc: upper/lower body venules, upper/lower body veins, superior/inferior vena cava.

The parameters that characterize the physiologic state of the closed-loop model are defined by each of the specific compartments, representing characteristics of the local area of circulation. For example, arterial and venous compartments are characterized by inertances, resistances and compliances. Cardiac chambers are characterized by activation functions which in turn are defined through elastances and activation times. Cardiac valves are characterized by parameters that model their ability to open and close. The model is able to describe physiologic signals over time, such as systemic arterial and venous pressure, pulmonary arterial and venous pressure, ventricle volumes, atrial volumes, cardiac valve flow rates, and compartment volumes. This set of variables is post-processed to obtain the hemodynamic profile (HP) of the virtual individual, which is represented by pressure-volume loops, mean, diastolic, and systolic pressures, cardiac output and index, and peak valve flow rates that characterize the virtual individual energetics such as aortic pulsatility index, ventricular and arterial elastances, coupling ratios, ventricular efficiency, ventricular stroke work, among others.

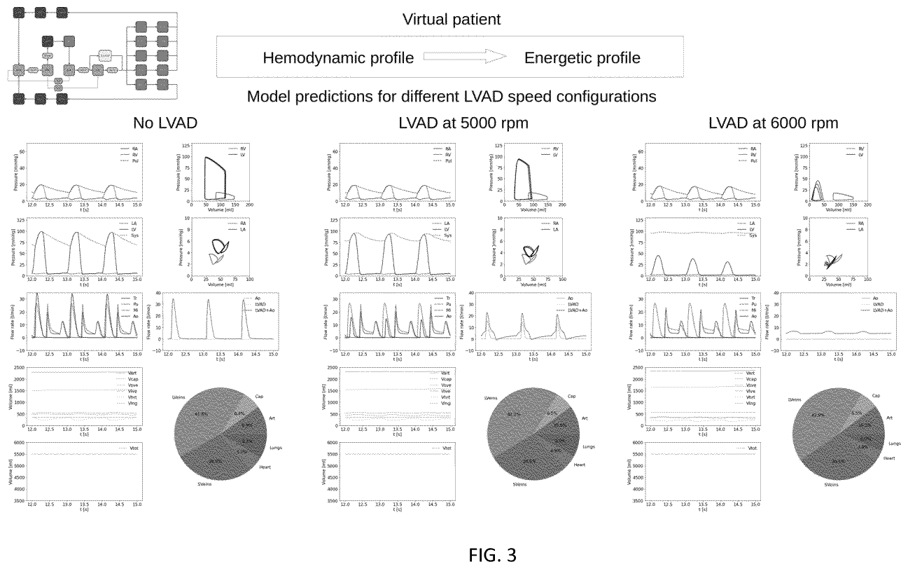

FIG. 3 is a schematic representation of a full hemodynamic profile generated by the closed-loop model shown in FIG. 2 for different LVAD working conditions. FIG. 3 illustrates the model capabilities in terms of physiologic signals (volume, flow rate and pressure waveforms) as well as the post-processing of these signals to deliver the characterization of the energetic state of the right and left ventricles through PV-loops, and blood volume distribution across body compartments. For each LVAD working condition (each one of the three major panels), the panels show the right atrial and pulmonary pressures (first row, left column), the left ventricle and systemic pressures (second row, left column), the flow rates through the four valves (third row, left column), the blood volume for each major compartment (fourth row, right column), the total volume in the system (fifth row, left column); the pressure-volume loop for the left and right ventricles (first row, right column); the pressure-volume loop for the left and right atria (second row, right column); the cardiac output (third row, right column); and the blood distribution across the body (pie chart, right column).

As an optional addition to further define the patient-specific energetic state, peripheral blood can be collected from each patient for metabolomic analysis. Loss of metabolic flexibility and a shift away from fatty acid and glucose oxidative metabolism is common with progressive heart failure and can provide additional prognostic information. Given the link between metabolic efficiency, mechanical efficiency of the heart and cardiac energetics, this added metabolomic information can incorporated into the modeling to further delineate the energetic state of the patient and improve on the risk stratification.

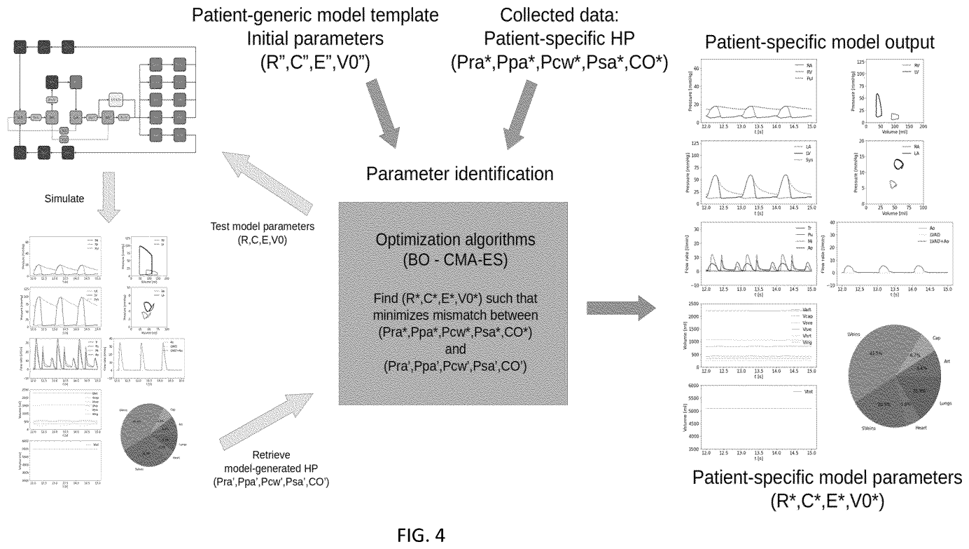

By modification of the parameters that characterize the above described closed-loop model of the cardiopulmonary circulation, it is possible to represent specific individuals. This stage is termed the parameter identification phase, and it is schematically represented in FIG. 4. To this end, the VPS is fed with the HP retrieved from a specific patient during a hemodynamic RHC. That is, mean atrial pressures, diastolic and systolic pulmonary and systemic pressures, cardiac output, and heart rate are collected, among other clinical variables. The aim of the parameter identification phase is to adjust the model parameters, specifically the systemic resistance and compliance, the pulmonary resistance and compliance, the venous resistance and compliance, the four-cardiac chamber baseline and maximum elastances, the reference chamber volume, and the blood volume, such that the HP of the closed-loop CVS model mimics the HP of the specific patient during the procedure. It will be appreciated that in some embodiments a subset of these parameters may be optimized. Likewise, embodiments that employ alternative VPS models may optimize different model-specific parameters. In this stage, the template of the cardiopulmonary circulation model is turned into a patient-specific model, also referred to as “constructing a digital twin of the patient,” which is one of the main roles of the VPS platform. By doing this, the platform described herein is capable of profiling a myriad of hidden phenomena that is happening in a patient behind the measured HP variables that are provided as input to the parameter identification procedure. That is, the VPS platform enables the enhancement of the HP given by the collected input data by providing an augmented hemodynamic state of the system according to the state variables (pressure, flow rate, volume) available in the different components of the model. Moreover, from this patient-specific model, the platform described herein is able to compute pressure-volume loops, peak valve flow rates, ventricular and arterial elastances, coupling ratios, ventricular efficiency, ventricular stroke work, among other variables which were not collected from the patient. These variables are referred to herein as hidden variables in the sense that they are not directly available in clinical measurements, but the model allows their characterization in order to verify the condition that the HP of the patient and that of the VPS match.

In the VPS platform, the parameter identification stage is carried out such that the mismatch between the HP of the cardiovascular (CVS) model, and that of the patient is minimized. This is accomplished by utilizing many statistical methods, for example but not limited to: (i) Bayesian optimization (BO); and (ii) covariance matrix adaptation evolution strategy (CMA-ES). These methods are employed whenever the function to be optimized is highly non-linear, non-convex, presents many local extrema, and is defined in a relatively high dimensional space. The dimension of the space is defined by the number of parameters that are to be optimized. In one example embodiment, up to 20 parameters are being optimized, and the optimization is carried out either for a specific patient (with a given HP), or for a given population (defined by a particular HP). Moreover, both BO and CMA-ES methods exploit the fact that the closed-loop model of the CVS is a black-box, and so the function evaluation that is to be minimized (i.e. the mismatch between the model HP and the patient HP) is also a black-box. Once initial in-depth VPS parameter optimization is established and a patient is represented virtually, the same virtual patient model could be re-calibrated based on a set of data that are more readily available, such as information from a smart watch or other wearable or implantable pressure monitors, to continuously update the model to reflect physiological status of the patient.

### Machine Learning for Heart Failure (ML4HF) Platform

The ML4HF platform relies on a set of machine learning models properly developed to handle patients with different stages of HF.

The current iteration of the ML4HF platform relies on retrospectively collected data from multiple sources, defining and creating a baseline algorithm that utilizes the methods described above for tuning. Iterative models will be self-learning in pathways that include:
Data preprocessing techniques that are applied to clean, normalize, and transform the data into a suitable format;
Feature engineering that involves extracting meaningful features using techniques like nearest neighbor and clustering to identify patterns and relationships within the dataset.
Association rules that are utilized to discover correlations between different variables.

In a model selection step, lightweight models like decision trees, random forests, or linear models are preferred for computational efficiency. Deep learning models such as neural networks may be employed for capturing complex patterns if necessary. Hyperparameter optimization fine-tunes the models, ensuring optimal performance.

Real-time prediction is facilitated by deploying the trained models in a live environment, where they receive incoming data and make predictions on-the-fly. Continuous model updating and retraining mechanisms are implemented to adapt to changing data patterns.

The testing and validation phase involves evaluating the models' performance using metrics like accuracy, precision, recall, and F1-score. Detailed validation procedures simulate real-world scenarios and edge cases to identify potential weaknesses and biases.

To provide optimal model performance and reproducibility with varied datasets, extensive testing is conducted. This includes:
Cross-Validation: Utilizing techniques such as k-fold cross-validation to evaluate the model's performance on multiple subsets of the data, ensuring robustness and generalization across different partitions.
Holdout Validation: Splitting the dataset into training and testing sets to assess the model's performance on unseen data, providing an estimate of its real-world performance.
Stratified Sampling: Ensuring that the distribution of classes in the training and testing datasets remains representative of the overall population, preventing biases in model evaluation.
Bootstrapping: Generating multiple bootstrap samples from the dataset to estimate the variability of model performance metrics and assess their stability across different samples.
Out-of-Distribution Testing: Evaluating the model's performance on data points that lie outside the distribution of the training data, identifying potential vulnerabilities and ensuring robustness in real-world scenarios.
A/B Testing: Comparing the performance of different model versions or algorithms in a controlled environment to determine the most effective approach.

The goal of the ML tools is to predict event outcomes based on the patient data at the baseline. This is accomplished in two stages.

### Stage 1

Stage 1 utilizes models within the pathways described below to predict the likelihood of a subject progressing to a point where advanced therapies may be required based on HP data.

Analyzing data within the ML4HF platform is conducted with various optimization techniques and iterative algorithm design. Functions at the core of the iteration and model refinement lead to convergence of outcomes and optimization demonstrated by ROCAUC. Functions important in this process are described below

### Gradient Descent

Recursive functions are fundamental in gradient descent optimization algorithms, such as stochastic gradient descent (SGD) and its variants. These functions iteratively update model parameters based on the gradient of the loss function with respect to the parameters.

At each iteration, the current parameter values are adjusted in the direction opposite to the gradient, aiming to minimize the loss function and converge towards the optimal solution.

The recursive nature of gradient descent involves repeatedly computing gradients and updating parameters until convergence criteria are met, such as reaching a specified tolerance level or completing a fixed number of iterations.

### Backpropagation

Backpropagation is a recursive algorithm used for training neural networks by efficiently computing gradients of the loss function with respect to the network's parameters.

The process involves recursively propagating error gradients backward through the network's layers, starting from the output layer and moving towards the input layer.

Recursive functions are employed to compute the gradients of intermediate layers by recursively applying the chain rule of calculus, which decomposes the gradient of the loss function into products of gradients at each layer.

### Recursive Feature Elimination (RFE)

RFE is a recursive feature selection technique used to identify the most relevant features for a given machine learning task.

It recursively removes features from the dataset, training the model on the remaining features and evaluating its performance.

Recursive functions are employed to iteratively eliminate less important features based on certain criteria, such as feature importance scores or coefficients obtained from the model.

### Recursive Partitioning

Decision tree-based algorithms, such as CART (Classification and Regression Trees) and Random Forests, utilize recursive partitioning techniques to split the data into subsets based on feature values.

Recursive functions are employed to recursively partition the data at each node of the tree, selecting the feature and split point that maximize information gain or minimize impurity.

This recursive process continues until certain stopping criteria are met, such as reaching a maximum tree depth or a minimum number of samples per leaf node.

These recursive functions play an important role in improving optimization in machine learning by iteratively refining model parameters, updating feature selection, and recursively partitioning data to converge towards optimal solutions. They enable efficient exploration of the solution space and facilitate the training of complex models on large datasets.

### Stage 2

Stage 2 is helps augment HP with energetic profile via VPS data related to the subject being analyzed. Predictive outputs here will be focused on the accuracy of events occurring in the future, including, but not limited to, heart transplantation, durable LVAD, or native organ recovery.

Both K-fold cross-validation and M-repeated validation are valuable techniques for training and validating machine learning models. They provide robust estimates of model performance, facilitate better generalization to unseen data, and help identify potential issues such as overfitting or data leakage. These methods can assess and compare the performance of different models and select the best-performing one for deployment.

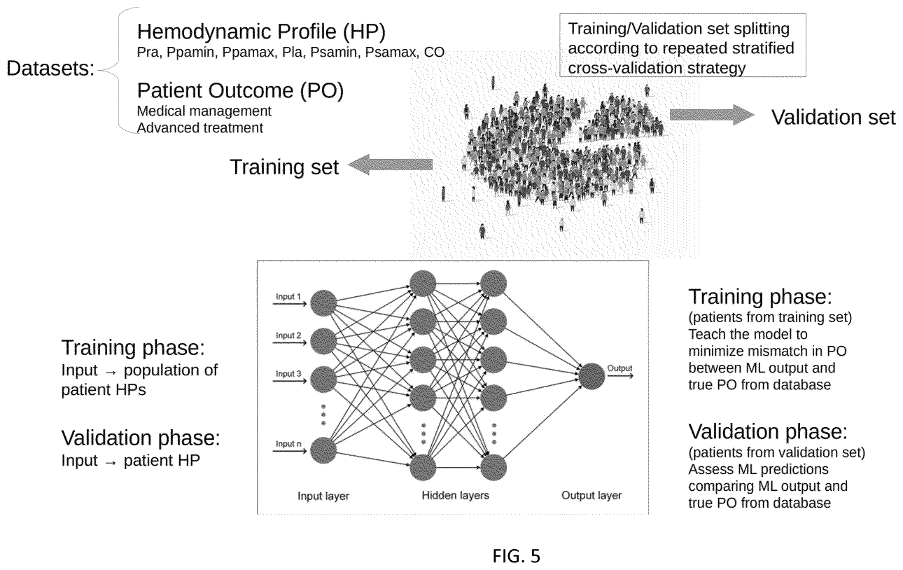

The method described herein leverages K-fold cross-validation and M-repeated validation techniques to effectively train and validate machine learning models using available datasets. Initially, the dataset is divided into K equally sized folds, with the model trained K times, iteratively using K-1 folds for training and the remaining fold for validation. This process ensures a comprehensive evaluation of the model's performance, enhancing its generalization ability. Additionally, M-repeated validation extends this approach by repeating the K-fold cross-validation process M times, enabling a thorough assessment across multiple random data partitions. By averaging the performance metrics obtained from each repetition, the method described herein delivers a stable and reliable estimate of the model's performance, aiding in model selection and optimization. These techniques contribute to the robustness and effectiveness of our approach in handling available datasets for machine learning tasks. The resultant output will be to predict the need for advanced therapies or stability with standard practices of medical care. FIG. 5 is a schematic representation of this process. FIG. 5 is a schematic representation of training and validation of an ML model, featuring input variables (patient hemodynamic profile) and endpoint patient outcome.

### Integrated VPS-ML4HF Platform

The integrated VPS-ML4HF platform aims at improving the predictive capabilities of the ML4HF models by incorporating into the input data array variables which are not collected from the patients because of clinical limitations. Thus, for each patient, the VPS is employed to create a patient-specific model by optimizing its parameters and create a personal precision based energetic profile of that patient. This combination of HP-EP is fed into the corresponding ML model to predict the outcome of advanced heart failure therapeutics (e.g., heart transplant, durable LVAD, cardiac recovery, or continuous intravenous medical therapy). The augmentation of the HP by the EP provides the ML model with (model-based) hidden information of the patient's state and allows to improve the overall predictive capabilities of the ML model.

The integration of VPS and ML4HF technologies requires an additional step in which the model is trained and validated using HP and EP (from model generated by parameter identification) profiles. This stage is computationally intensive as it requires parameter identification for all the patients in the databases. Once the pairs of HP/EP have been generated, the M-repeated stratified K-fold cross-validation approach is exploited to build the ML models (As described above).

An ensemble of these models is generated to predict the outcome of any given new patient. The assessment of any new patient starts with collecting the patient HP. Then, the patient-specific parameter identification is carried out, which delivers the model-generated EP. Then, the pair HP/EP is given as input into the ML model, which predicts the probability that the patient needs advanced heart failure therapies.

To optimize training, virtual populations can be augmented with specified HP from the VPS. In order to perform this customized approach, unique to the platform described herein, a range is set within which model parameters are to be modified, and the VPS is utilized to generate, for each set of model parameters, a single virtual patient. This patient has both HP and EP generated by the model. This data augmentation procedure helps in the regularization of the ML models. It adds physiologically and energetically consistent data into the training phase. Thus, using the VPS it is possible to can set virtual patients featuring different levels of disease stage, based on the specifications of the HP, and then train the ML model under a wide range of disease conditions. The platform described herein also allows the possibility to exploit the VPS in generating virtual patients to explore the predictive limitations of the ML model.

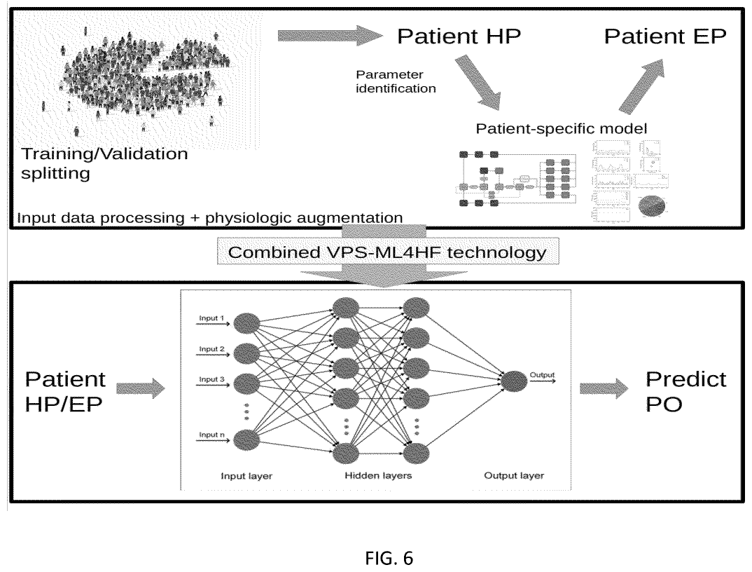

The ML platform escribed herein employs virtual or simulated data to predict real-world outcomes through a process involving digital twinning and retrospective analysis, schematically represented in FIG. 6. Initially, a digital twin environment is created, mirroring real-world scenarios and interactions. This virtual environment generates simulated data that closely resembles real-world observations. By correlating events and outcomes in the digital twin environment with actual outcomes retrospectively, the platform described herein learns patterns and relationships between simulated data and real-world outcomes. In the exemplary embodiment shown in FIG. 6, patient HP is augmented using the CVS model to find variables that characterize the hidden energetic state of the patient. The pair HP/EP is fed to the ML model to predict patient outcome (PO).

Once correlations are established, our platform modifies the predictive model to forecast future outcomes in real-time. This involves leveraging a baseline set of real-world data to refine the model's predictions continuously. The modified model is trained on historical data, including observations from the digital twin environment and real-world outcomes, to capture evolving patterns and dynamics accurately.

To predict future outcomes in real-time, the model analyzes incoming data streams from the real world, compares them with simulated data from the digital twin, and adjusts predictions accordingly. This adaptive approach allows our platform to anticipate changes and mitigate risks effectively. By integrating digital twinning, retrospective analysis, and real-time prediction, the ML platform described herein offers a robust solution for forecasting real-world outcomes and adapting to dynamic environments.

Additionally, correlation analysis and mutual information estimation are employed to unveil intricate relationships between simulated events and real-world outcomes, ensuring a comprehensive understanding of data dynamics.

In various embodiments, systems described herein are implemented in a SAAS model in which an output is provided from a central system based on parameters transmitted thereto. Accordingly, such embodiments may be regarded as employing an EMR/data-to-cloud model. In other embodiments, a System-On-Chip (SOC) is provided that is integrated with or connectable to a cardiac monitoring system to provide local data analysis and feedback.

### ML4HF, VPS and SOC Integration

The system and method disclosed herein offer a comprehensive solution for integrating real-time predictive analytics algorithms with System-on-Chip (SOC) architecture, ensuring scalability, efficiency, and adaptability across various applications. The SOC architecture comprises a highly integrated system incorporating processing units, memory, and peripheral interfaces on a single chip, enabling seamless execution of diverse computational tasks.

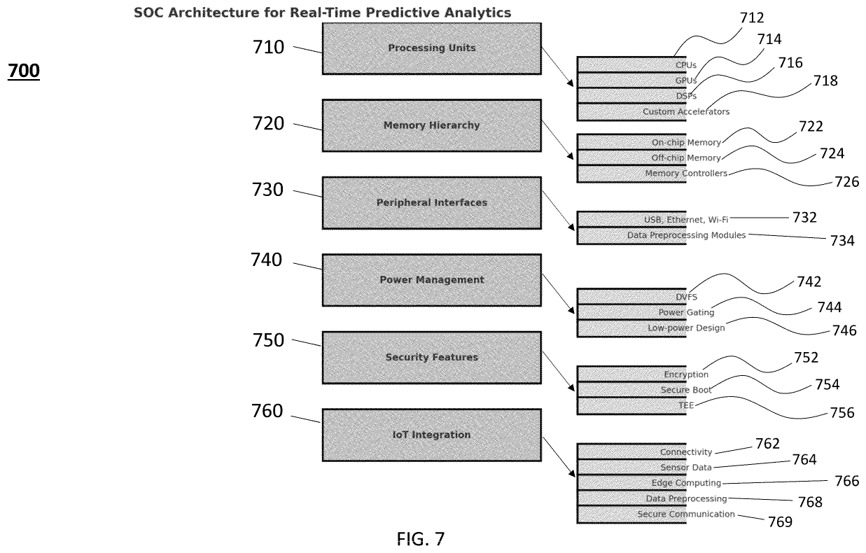

An exemplary SOC architecture 700 tailored for real-time predictive analytics is shown in FIG. 7.

SoC 700 can comprise one or more Processing Units 710. The SOC 700 can integrate, for example high-performance processing units such as Central Processing Units (CPUs) 712, Graphics Processing Units (GPUs) 714, or specialized accelerators like Digital Signal Processors (DSPs) 716 optimized for machine learning tasks. Custom instruction sets and hardware accelerators 718 can be designed to accelerate common machine learning operations such as matrix multiplications and activation functions, enhancing computational efficiency.

Soc 700 can comprise Memory Hierarchy 720. The SOC 700 can incorporate a hierarchical memory architecture comprising fast on-chip memory 722 (e.g., caches) and off-chip memory 724 (e.g., DRAM), optimized for low-latency data access and efficient storage of model parameters and intermediate data. Memory controllers 726 and cache coherence protocols can be designed to maximize memory bandwidth and minimize data movement overhead during computation.

SoC 700 can comprise Peripheral Interfaces 730. The SOC 700 can feature versatile peripheral interfaces such as Universal Serial Bus (USB), Ethernet, Wi-Fi, Bluetooth 732, and various sensor interfaces, enabling seamless integration with external devices and data sources. Dedicated hardware modules 734 can be designed to handle data preprocessing, sensor fusion, and real-time data acquisition, facilitating smooth data flow into the predictive analytics pipeline.

Soc 700 can comprise Power Management 740. The SOC 700 can implement advanced power management techniques such as dynamic voltage and frequency scaling (DVFS) 742, power gating 744, and/or clock gating (not shown) to optimize energy efficiency while meeting real-time performance requirements. Low-power design methodologies 746 can be employed to minimize energy consumption during idle and low-load periods, extending battery life in portable and internet-of-things (IoT) devices (see below with respect to element 760).

SoC 700 can comprise Security Features 750. Robust security features such as hardware-based encryption 752, secure boot 754, and trusted execution environments (TEEs) 756 can be integrated into the SOC to safeguard sensitive data and ensure the integrity of predictive analytics algorithms and models. Secure communication protocols and access control mechanisms are implemented to protect data privacy and prevent unauthorized access to system resources.

SoC 700 can also comprise at least one IoT Integration Node 760 providing integration to the Internet-of-Things (IoT). The IoT Integration Node 760 can comprise the following modules.

Node 760 can incorporate connectivity module 762 providing versatile communication interfaces such as Wi-Fi, Bluetooth Low Energy (BLE), Zigbee, and LoRaWAN, enabling seamless connectivity with a wide range of IoT devices. Protocol stacks and communication protocols are optimized for low-power, low-latency data transmission, ensuring efficient data exchange between the SOC and IoT devices.

Node 760 can comprise Sensor Data Acquisition and Fusion module 764. Module 764 can provide dedicated hardware modules within the SoC 700 designed to interface with various sensors commonly found in IoT devices, including temperature sensors, humidity sensors, accelerometers, and environmental sensors. Advanced sensor fusion algorithms are implemented to aggregate and process data from multiple sensors, providing rich contextual information for predictive analytics tasks.

Node 760 can comprise Edge Computing module 766. The SoC architecture 700 can incorporate edge computing capabilities, enabling on-device processing and analysis of sensor data in real-time. Machine learning inference engines optimized for low-power consumption are integrated into the SoC 700, allowing predictive analytics algorithms to run directly on IoT devices without relying on cloud-based processing.

Node 760 can comprise Data Preprocessing and Feature Extraction module 768. Module 768 can perform data cleaning, normalization, and feature extraction tasks on sensor data streams before feeding them into the predictive analytics pipeline. Custom hardware accelerators and firmware libraries are developed to accelerate computationally intensive preprocessing tasks, ensuring real-time responsiveness and efficient resource utilization.

Node 760 can comprise Secure Communication and Data Privacy module 769. Secure communication protocols such as Transport Layer Security (TLS) and Datagram Transport Layer Security (DTLS) can be implemented to establish encrypted communication channels between the SOC and IoT devices, ensuring data privacy and integrity. Access control mechanisms and authentication protocols are enforced to prevent unauthorized access to sensitive data and resources within the IoT ecosystem.

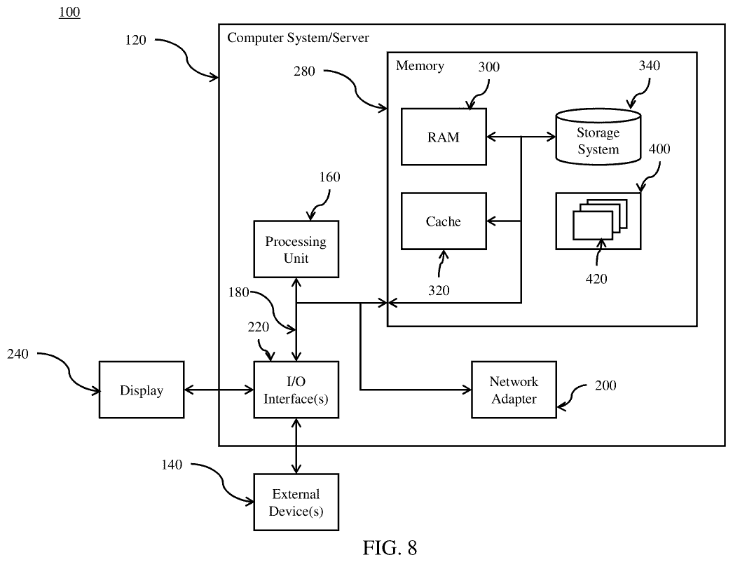

In various embodiments, the devices described herein, including any of the nodes and modules described above with respect to SoC 700 can comprise at least one computer node 100 as shown in FIG. 8.

As shown in FIG. 8, computer system/server 120 in computing node 100 is shown in the form of a general-purpose computing device. The components of computer system/server 120 may include, but are not limited to, one or more processors or processing units 160, a system memory 280, and a bus 180 that couples various system components including system memory 280 to processor 160.

Bus 180 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 120 typically includes a variety of computer system readable media. Such media may be any available media that is accessible by computer system/server 120, and it includes both volatile and non-volatile media, removable and non-removable media.

System memory 280 can include computer system readable media in the form of volatile memory, such as random access memory (RAM) 300 and/or cache memory 320. Computer system/server 120 may further include other removable/non-removable, volatile/non-volatile computer system storage media. By way of example only, storage system 340 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 180 by one or more data media interfaces. As will be further depicted and described below, memory 280 may include at least one program product having a set (e.g., at least one) of program modules that are configured to carry out the functions of embodiments of the disclosure.

Program/utility 400, having a set (at least one) of program modules 420, may be stored in memory 280 by way of example, and not limitation, as well as an operating system, one or more application programs, other program modules, and program data. Each of the operating system, one or more application programs, other program modules, and program data or some combination thereof, may include an implementation of a networking environment. Program modules 420 generally carry out the functions and/or methodologies of embodiments as described herein.

Computer system/server 120 may also communicate with one or more external devices 140 such as a keyboard, a pointing device, a display 240, etc. ; one or more devices that enable a user to interact with computer system/server 120; and/or any devices (e.g., network card, modem, etc.) that enable computer system/server 120 to communicate with one or more other computing devices. Such communication can occur via Input/Output (I/O) interfaces 220. Still yet, computer system/server 120 can communicate with one or more networks such as a local area network (LAN), a general wide area network (WAN), and/or a public network (e.g., the Internet) via network adapter 200. As depicted, network adapter 200 communicates with the other components of computer system/server 120 via bus 180. It should be understood that although not shown, other hardware and/or software components could be used in conjunction with computer system/server 120. Examples, include, but are not limited to: microcode, device drivers, redundant processing units, external disk drive arrays, RAID systems, tape drives, and data archival storage systems, etc.

Accordingly, in a first example embodiment, the present invention is a method. In a 1^{st} aspect of the 1^{st} example embodiment, the method comprises: reading a hemodynamic profile of a patient; based on the hemodynamic profile, tuning a plurality of parameters of a cardiovascular model to create a digital twin of the patient; augmenting the hemodynamic profile of the patient with at least one parameter generated from the digital twin; providing the augmented hemodynamic profile to a pretrained machine learning model and receiving therefrom a patient profile; and outputting the patient profile for clinical decision support.

In a 2^{nd} aspect of the 1^{st} example embodiment, the hemodynamic profile consists essentially of clinically measured parameters of the patient. The remainder of the features and example features are as described above with respect to the 1^{st} aspect of the 1^{st} example embodiment.

In a 3^{rd} aspect of the 1^{st} example embodiment, the hemodynamic profile comprises one or more of: mean atrial pressures, diastolic and systolic pulmonary and systemic pressures, cardiac output, and heart rate. The remainder of the features and example features are as described above with respect to the 1^{st} through 2^{nd} aspects of the 1^{st} example embodiment.

In a 4^{th} aspect of the 1^{st} example embodiment, the cardiovascular model comprises a closed-loop compartmental representation. The remainder of the features and example features are as described above with respect to the 1^{st} through 3^{rd} aspects of the 1^{st} example embodiment.

In a 5^{th} aspect of the 1^{st} example embodiment, the compartmental representation comprises compartments corresponding to a systemic arterial district, a pulmonary arterial district, a venous pool, a right atrium, a left atrium, a right ventricle, and left ventricle. The remainder of the features and example features are as described above with respect to the 1^{st} through 4^{th} aspects of the 1^{st} example embodiment.

In a 6^{th} aspect of the 1^{st} example embodiment, the compartmental representation comprises a compartment corresponding to a mechanical support device. The remainder of the features and example features are as described above with respect to the 1^{st} through 5^{th} aspects of the 1^{st} example embodiment.

In a 7^{th} aspect of the 1^{st} example embodiment, each compartment has associated state values including blood pressure, blood flow rate, and blood volume. The remainder of the features and example features are as described above with respect to the 1^{st} through 6^{th} aspects of the 1^{st} example embodiment.

In an 8^{th} aspect of the 1^{st} example embodiment, the at least one parameter generated from the digital twin comprises a pressure volume loop. The remainder of the features and example features are as described above with respect to the 1^{st} through 7^{th} aspects of the 1^{st} example embodiment.

In a 9^{th} aspect of the 1^{st} example embodiment, the at least one parameter generated from the digital twin comprises cardiac energetics and/or coupling ratio. The remainder of the features and example features are as described above with respect to the 1^{st} through 8^{th} aspects of the 1^{st} example embodiment.

In a 10^{th} aspect of the 1^{st} example embodiment, the at least one parameter generated from the digital twin comprises one or more of: peak valve flow rates, ventricular and arterial elastances, coupling ratios, ventricular efficiency, or ventricular stroke work. The remainder of the features and example features are as described above with respect to the 1^{st} through 9^{th} aspects of the 1^{st} example embodiment.

In an 11^{th} aspect of the 1^{st} example embodiment, the patient profile comprises one or more of: acuity of illness, aggressiveness of needed therapy, likelihood of progression, and likely outcome. The remainder of the features and example features are as described above with respect to the 1^{st} through 10^{th} aspects of the 1^{st} example embodiment.

In a 12^{th} aspect of the 1^{st} example embodiment, tuning the plurality of parameters comprises Bayesian optimization or covariance matrix adaptation evolution. The remainder of the features and example features are as described above with respect to the 1^{st} through 11^{th} aspects of the 1^{st} example embodiment.

In a 13^{th} aspect of the 1^{st} example embodiment, the method further comprises the steps of: continuously monitoring the patient to update the hemodynamic profile; and retuning the plurality of parameters of the cardiovascular model. The remainder of the features and example features are as described above with respect to the 1^{st} through 12^{th} aspects of the 1^{st} example embodiment.

In a 14^{th} aspect of the 1^{st} example embodiment, the pretrained machine learning model comprises a decision tree, random forest, linear model, or artificial neural network. The remainder of the features and example features are as described above with respect to the 1 st through 13^{th} aspects of the 1^{st} example embodiment.

In a 2^{nd} example embodiment, the present invention is a system. In a 1^{st} aspect of the 2^{nd} example embodiment, the system comprises at least one cardiac sensor adapted to measure a hemodynamic profile of a patient; and a computing node operatively coupled to the cardiac sensor and configured to perform a method according to the 1^{st} example embodiment and any of its aspects.

In a 3^{rd} aspect, the present invention is a computer program product for clinical decision support. In a 1^{st} aspect of the 3^{rd} example embodiment, the computer program product comprises a computer readable storage medium having program instructions embodied therewith, the program instructions executable by a processor to cause the processor to perform a method according to the 1^{st} example embodiment and any of its aspects.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method comprising:
reading a hemodynamic profile of a patient;
based on the hemodynamic profile, tuning a plurality of parameters of a cardiovascular model to create a digital twin of the patient;
augmenting the hemodynamic profile of the patient with at least one parameter generated from the digital twin;
providing the augmented hemodynamic profile to a pretrained machine learning model and receiving therefrom a patient profile; and
outputting the patient profile for clinical decision support.

2. The method of claim 1, wherein the hemodynamic profile consists essentially of clinically measured parameters of the patient.

3. The method of claim 1, wherein the hemodynamic profile comprises one or more of:
mean atrial pressures, diastolic and systolic pulmonary and systemic pressures, cardiac output, and heart rate.

4. The method of claim 1, wherein the cardiovascular model comprises a closed-loop compartmental representation.

5. The method of claim 4, wherein the compartmental representation comprises compartments corresponding to a systemic arterial district, a pulmonary arterial district, a venous pool, a right atrium, a left atrium, a right ventricle, and left ventricle.

6. The method of claim 4, wherein the compartmental representation comprises a compartment corresponding to a mechanical support device.

7. The method of claim 5 or 6 wherein each compartment has associated state values including blood pressure, blood flow rate, and blood volume.

8. The method of claim 1, wherein the at least one parameter generated from the digital twin comprises a pressure volume loop.

9. The method of claim 1, wherein the at least one parameter generated from the digital twin comprises cardiac energetics and/or coupling ratio.

10. The method of claim 1, wherein the at least one parameter generated from the digital twin comprises one or more of: peak valve flow rates, ventricular and arterial elastances, coupling ratios, ventricular efficiency, or ventricular stroke work.

11. The method of claim 1, wherein the patient profile comprises one or more of: acuity of illness, aggressiveness of needed therapy, likelihood of progression, and likely outcome.

12. The method of claim 1, wherein tuning the plurality of parameters comprises Bayesian optimization or covariance matrix adaptation evolution.

13. The method of claim 1, further comprising:
continuously monitoring the patient to update the hemodynamic profile; and
retuning the plurality of parameters of the cardiovascular model.

14. The method of claim 1, wherein the pretrained machine learning model comprises a decision tree, random forest, linear model, or artificial neural network.

15. A system comprising:
at least one cardiac sensor adapted to measure a hemodynamic profile of a patient; and
a computing node operatively coupled to the cardiac sensor and configured to perform a method according to any one of claims 1 to 14.

16. A computer program product for clinical decision support, the computer program product comprising a computer readable storage medium having program instructions embodied therewith, the program instructions executable by a processor to cause the processor to perform a method according to any one of claims 1 to 14.
